(19) United States
(12) Patent Application Publication
(10) Pub. No.: US 2009/0098591 A1
(43) Pub. Date: Apr. 16, 2009

(54) Method of Correlating Marker Molecule Concentration To A Specific Cell Potency Level in Chondrocyte Culture

(76) Inventors: Wiltrud Richter, Neckargemuend (DE); Karollina Pelttari, Basel (CH); Stephane Boeuf, Heidelberg (DE)

Correspondence Address: Lauson & Tarver LLP, 880 Apollo Street, SUITE 301, El Segundo, CA, 90245, US

(21) Appl. No.: 12/201,511

(22) Filed: Aug. 29, 2008

(30) Foreign Application Priority Data
Jun. 14, 2006 (DE) 10-2007-027-991A1
Jun. 14, 2007 (EP) 1-873-236 A1

(51) Int. Cl. C12Q 1/37; C12Q 1/02

(52) U.S. Cl. 435/24; 435/29

(57) ABSTRACT

A method for measuring the potency of tissue culture derived chondrocyte or chondrocyte-like cells by correlating levels of the marker molecules SERPINA1 and MMP3 in culture supernatant with a specific level of cell potency; including a method for analyzing chondrogenic potency in cells re-differentiated from chondrocytes losing their chondrogenic potency, or which are differentiated from cells to receive chondrogenic potency; comprising the steps of ascertaining the correlative value of the level of SERPINA1, MMP3, or SERPINA1 and MMP3 marker molecules with the potency level of cultured chondrogenic or chondrogenic-like cells; measuring the level of SERPINA1, MMP3, or SERPINA1 and MMP3 marker molecules contained in the supernatant of monolayer or 3D matrix cultured chondrogenic or chondrogenic-like cells; and ascertaining the potency level of the cells from the concentration of the marker molecules in the measurement.

### CROSS-REFERENCE TO RELATED APPLICATION

This application claims the benefit of the priority dates of European patent application number 1-873-236 A1, filed on Jun. 14, 2007, which claims the benefit of the priority date of German patent application number 10-2007-027-991 A1, filed on Jun. 14, 2006.

### FEDERALLY SPONSORED RESEARCH

Not Applicable

### SEQUENCE LISTING OR PROGRAM

Not Applicable

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

Cartilage shows a very limited capacity for tissue regeneration. Therefore in medical transplantation engineering, there has been an interest in substituting cartilage with autologous cells, including autologous cells having the potential for differentiation to cartilage. This interest has contributed to increased study into tissue culture systems, where tissues can be controlled and monitored during their differentiation states as they form cartilage.

Of particular interest is the optimization of expansion and differentiation of cultured human and animal chondrocytes, since cartilage tissue engineering and newly developed cell therapeutic methods such as autologous chondrocyte transplantation (ACT) rely on chondrocytes or chondrocyte-like cells obtaining expanded chondrogenic potency in tissue culture systems.

In ACT, small cartilage biopsies are taken from lesser impacted regions of a patient's joint; particular areas include injured cartilage of the knee or vertebra. After the biopsy process, chondrocytes embedded in the extracellular matrix of cartilage tissue are released by enzymatic digestion of the matrix. After digestion, the chondrocytes obtained are expanded in a cell culture medium, including grown stimulation factors in order to obtain a high cell number count for transplantation.

In addition, mesenchymal stem cells (MSC), which are also autologous, fall into the category of chondrocyte-like cells. Mesenchymal stem cells are an attractive alternative cell source from which to obtain chondrocytes for an ACT process, since they can be isolated from diverse tissues without irreversible tissue damage.

De-differentiation of chondrocytes and a low rate of proliferation during cell culture results in a poor transplantation success rate. Since monolayer culturing increases chondrocyte proliferation capacity and decreases de-differentiation, it is the chosen method of tissue engineering for generating chondrocytes used in ACT. In addition to the native effects of monolayer culturing, growth factors are used to initiate re-differentiation of chondrocytes or for chondrogenic differentiation of MSC in vitro.

Due to the risk of cell leakage, uneven cell distribution within the injured cartilage, and hypertrophy of the repair tissue after injecting chondrocytes in suspension beyond a sutured periosteal flap, matrix culture techniques have been developed. For example, embedding cells in three dimensional scaffolds can facilitate transplantation. These three dimensional scaffolds, so-named because of the three dimensionality of their culture systems or models, can be made of solid porous material or hydrogels.

When using the techniques mentioned above, controlling the differentiation state and capacity of the tissues to form cartilage is required. To determine the differentiation state, some of the cells must be sacrificed from the monolayer culture or three dimensional scaffolding for DNA and/or RNA isolation. This process can be very time consuming, and the sacrificed cells must be replaced by culturing remaining cells. This makes the prognosis for any given transplantation very difficult to ascertain, since the differentiation state of the cultured cells can't be determined at the time of transplantation.

Therefore, there is a need for a method of analyzing chondrocytes during the re-differentiation process in vitro without sacrificing cells. Using such a method, cells that are no longer appropriate for implantation (i.e., no longer having chondrogentic potency), but which can be treated with special factors, such as growth factors to re-differentiate them to cartilage regenerating cells can be identified. A protocol for re-differentiation can be ascertained by collecting and measuring data. This method can be an alternative for picking out de-differentiated cells that have lost the capacity for forming cartilage from cell culture.

### SUMMARY

The present invention provides a method for measuring SERPINA1 and/or MMP3 as secreted marker molecules from cells in monolayer culture systems or from cells embedded in three dimensional scaffolds for correlation with a concentration of these marker molecules with a specific level of cell potency.

In particular, the present invention provides a method for measuring the concentration of SERPINA1 or MMP3 or a combination of concentrations of these markers in the supernatant of the cultured chondrocytes or chondrocyte-like cells in monolayer culture systems or from cells embedded in three dimensional scaffolds.

The invention further establishes a method for analyzing chondrogenic potency in cells re-differentiated from chondrocytes losing their chondrogenic potency or which are differentiated from cells to receive chondrogenic potency by measuring SERPINA1 and/or MMP3 as secreted marker molecules from cells in a growth factor treated culture. The method also comprises establishing a condrogenic re-differentiation protocol for returning chondrogenic potency to chrondocytes as well as establishing a chondrogenic differentiation protocol for cells achieving chondrogenic potency.

### DETAILED DESCRIPTION

The present invention comprises a method for measuring SERPINA1 and/or MMP3 as secreted marker molecules from cells in monolayer culture for correlation of the concentration of these markers with a specific cell potency. The culture supernatant is isolated from a cultivation of cell cultures in a liquid medium.

The SERPINA1 gene provides instructions for making a protein called alpha-1 antitrypsin, which is a type of serpin protein. Serpin proteins help control several types of chemical reactions by inhibiting certain enzymes. Alpha-1 antitrypsin is named for inhibiting properties against the digestive enzyme trypsin. It also inhibits other enzymes including neutrophil elastase, a powerful enzyme found in white blood cells. Therefore, the marker molecule SERPINA1 measured in the cell culture supernatant is the aforementioned serpin protein or a part of this protein secreted from the cultured cells into the culture supernatant.

MMP3 is known as Matrix metalloproteinase 3 (stromelysin 1, progelatinase). This protein is involved in the breakdown of extracellular matrix in normal physiological processes, such as reproduction and tissue remodeling. The protein is secreted as inactive proproteins which are activated when cleaved by extracellular proteinases. The MMP3 gene encodes an enzyme which degrades fibronectin, laminin, collagens III, IV, IX, and X, and cartilage proteoglycans. Therefore, the marker molecule measured in the cell culture supernatant is the aforementioned enzyme or a part of this enzyme.

The term “secreted marker molecules” refers to molecules secreted from cells in liquid medium cultivation. The secreted marker molecules described in this invention are SERPINA1 and MMP3 that allow monitoring of expansion and differentiation in vitro and thus comparison and optimization of cell cultures for in vivo applications

The term “correlating” used with reference to the concentration of the marker molecules indicates a correlation between the measured marker molecules secreted from the cells into the supernatant of the liquid culture medium and the chondrogenic potency of the cells.

The term “combination of the concentrations” refers to an advanced specificity if both markers SERPINA1 and MMP3 are measured simultaneously to ascertain chondrogenic potency in comparison to measuring only one marker.

All chondrocytes described herein are mammal chondrocytes found in human or in animal cartilage which produce and maintain the cartilaginous matrix, which consists mainly of collagen and proteoglycans.

“Chondrocyte-like cells” of the invention are cells which can terminally differentiate into chondrocytes. For example, colony-forming unit-fibroblasts (CFU-F) or mesenchymal stem cells are chondrocyte-like cells for purposes of this invention.

The term “monolayer culture systems” refers to a layer of cells in which no cell is growing on top of another, but all are growing side by side and often touching each other on the same growth surface.

The term “cells embedded in three dimensional scaffolds” refers to cells seeded in a three dimensional structure or seeded in a gel or gel-like matrix to form a transplantation body.

The term “chondrogenic potency” used in this application describes cells having the capacity to form substitute cartilage when implanted by injection or transplanted in a three dimensional scaffold into a point of defect in the cartilage.

The term “re-differentiation” refers to molecular effects in chondrocytes that have lost their chondrogenic potency, assumed a fibroblast-like morphology and have ceased to synthesize cartilage-specific molecules like collagen 2 and aggrecan. However these effects are reversible and chondrocytes capable of re-differentiation can get back their chondrogenic potency with growth factor treatment. The term “re-differentiation” when used in the invention also refers to the acceleration of the redifferentiation process by up-regulation of chondrocyte-specific genes using specific growth factors.

The term “chondrocytes losing their chondrogenic potency” refers to chondrocytes in monolayer or three-dimensional culture systems showing changes that include: loss of their rounded cell shape, loss of collagen type 11 and aggrecan core protein expression, and loss of the capacity to induce stable cartilage implants after transplantation into a defective area of cartilage.

The term “differentiated from cells to receive chondrogenic potency” refers to chondrocyte-like cells as explained above expanded in vitro in cell culture systems. For example, when referring to cartilage, mesenchymal stem cells (MSC) are commonly known as chondrogenic cells since MSC have shown the ability to differentiate into osteoblasts. In vivo, differentiation of MSC in a non-vascularized area (such as cartilage) yields a chondrocyte.

The term “growth factor treated culture” refers to cell cultures in monolayer or three dimensional culture system conditions treated with recombinant growth factors stimulating de-differentiation, re-differentiation or differentiation of cells into cells with chondrogenic potency by directed gene expression or gene inhibiting, for example the stimulation of proteoglycan synthesis by BMP-4.

The term “growth factor” used herein also refers to hormones and morphogenes. In particular: BMP (bone morphogenic proteins) like BMP-2, BMP-4, BMP-6 and BMP-7; TGF-β (transforming growth factor beta); and IGF-1 (insulin-like growth factor 1) have an effect on the proteoglycan synthesis of chondrocytes.

The term “protocol” refers to standardized data to determine the capacity of chondrocytes to proliferate, altered gene expression in chondrocytes or the chondrogenic potency of re-differentiated chondrocytes, or the differentiation state of chondrocyte-like cells with chondrogenic potency switched to chondrocytes.

The practice of the invention involves culture models including monolayer, three dimensional culture systems such as alginate culture or high density pellet culture, and explant models. Tissue engineering techniques to achieve these culture models are known in the art. A wide variety of expansion protocols of different mammalian cell types such as by application of growth factors are also well-known to persons of skill.

Cartilage samples from peripheral, less load bearing regions of the knee joints were obtained after informed consent from parties undergoing total joint replacement in an ethically approved study. Chondrocyte isolation and culture was performed by removing cartilage slices outside regions with macroscopic evident degeneration from the underlying bone, mincing the slices with a scalpel, and digesting with 1.5 mg/ml of collagenase B (Roche, Mannheim, Germany) and 0.1 mg/ml hyaluronidase (Serva, Heidelberg, Germany) overnight (18 h) at 37° C. The released cells were plated in 80 cm² cell culture flasks in Dulbeccos modified Eagle's medium (DMEM (Life Technologies, Karlsruhe, Germany)) supplemented with 10% fetal calf serum (FCS), 100 U/ml penicillin, 100 μg/ml streptomycin, and maintained in a humidified atmosphere at 6% CO₂ and 37° C.

The medium was replaced twice a week. Confluence was achieved after 2-3 weeks. For measuring the concentration of SERPINA1 and MMP3, supernatant from the medium was taken at different points in time. For correlating the concentration of these markers with cell specific potency, RNA and cDNA was isolated from cultured cells by the methods described in W. Richter et al. (2002) Biochemical and Biophysical Research Communications 293: 284-292.

Alternatively, the isolated cells were encapsulated in alginate beads as three dimensional scaffolds at a density of 1×10⁶ cells/ml of gel as described in E. J. Thonar et al. (1994) J. Cell Sci., 107 (Pt1): 17-27. Briefly, cells were suspended in sterile 0.15 M NaCl containing low viscosity alginate gel (1.2%) and then slowly pressed through a 22 gauge needle in a drop-wise fashion into a 102 mM CaCl₂ solution.

After instantaneous gelation, the beads were maintained in a complete medium in a humidified atmosphere at 6% CO₂ and 37° C. The medium was replaced twice a week. For measuring the concentration of SERPINA1 and MMP3, supernatant from the medium was taken at different points in time. For correlating the concentration of these markers with cell specific potency, RNA and cDNA were isolated from cultured cells by the methods described in W. Richter et al. (2002) Biochemical and Biophysical Research Communications 293: 284-292.

Lipoaspirates generated during liposuction procedures were obtained from Proaesthetic Private Clinic, Heidelberg as described in W. Richter et al. (2008), Frontiers at Bioscience (13): 4517-4528.

For expansion, adipose tissue-derived MSC were plated at a cell density of 1-6×10³ cells/cm² in monolayer cultures in DMEM or alternatively in a gel as a three dimensional scaffold after chondrogenic differentiation of MSC in standard culture with 10 ng/ml recombinant human transforming growth factor beta 3 (TGF-beta 3) and 10 ng/ml recombinant human bone morphogenic protein 6 (BMP-6, R&D Systems, Wiesbaden, Germany), see W. Richter et al. (2008), Frontiers at Bioscience (13): 4517-4528. For measuring the concentration of SERPINA1 and MMP3, supernatant from the medium was taken at different points in time. For correlating the concentration of these markers with cell specific potency, RNA and cDNA were also isolated from cultured cells by the methods described in W. Richter et al. (2002) Biochemical and Biophysical Research Communications 293: 284-292.

At different points in time; PD0: Chondrocytes or MSC in monolayer culture or in a three dimensional scaffold culture with no expansion (no population germination), PD2: after two population germinations, PD6: after 6 population germinations, and at the transplantation point (PDtp), supernatant from the medium was taken as described in EP 1 873 236 A1, filed Jun. 14, 2007. At these points in time (PD0, PD2, PD6 and PDtp), cells were also harvested for preparing RNA and cDNA. Gene expression levels of chondrocytes were performed as reported in W. Richter et al. (2002) Biochemical and Biophysical Research Communications 293: 284-292.

Gene expression levels of MSC in monolayer culture and in three dimensional scaffold culture, determined as shown in W. Richter et al. (2008), Frontiers at Bioscience (13): 4517-4528, showed a decreased gene expression from time points PD0 to PDtp for SERPINA1 and MMP3.

Experiments with cDNA as described in W. Richter et al. (2002) Biochemical and Biophysical Research Communications 293: 284-292 for measuring the quantity of SERPINA1 and MMP3 showed lowering signal intensities in southern blot experiments from PD0 to PDtp.

The protein level of SERPINA1 and MMP3 obtained from the supernatant with an enzyme linked immunosorbent assay showed a reduction in the concentration of SERPINA1 and MMP3 in the supernatant of all culture systems.

The availability of SERPINA1 and MMP3 from the supernatant in cell culture systems provides a method for measuring chondrogenic potency. Correlations of the reduction in SERPINA1 and MMP3 concentrations in the supernatant of a culture medium with datasets from the RNA and cDNA experiments showed that gene expression of chondrocyte relevant RNA, and signal intensities of chondrocyte relevant cDNA were lowered.

So with this invention, chondrongenic potency can be reliably monitored without sacrificing cells to determine SERPINA1 and MMP3 levels secreted from cells in the medium; not only from cells suspended in a culture medium (see EP 873 236 A1, filed Jun. 14, 2007), but also from monolayer cell culture systems and from three dimensional culture systems. Therefore with the method claimed by this invention, chondrongenic potency in three dimensional scaffold cultures can be monitored at the point of transplantation.

With this invention, it is also possible to predict the success of a particular cartilage therapy by recording SERPINA1 and MMP3 before and during the term of cultivation as markers for the quality (chondrongenic potency) of three dimensional scaffold cultures without sacrificing cells. By measuring the concentrations of SERPINA1 and MMP3 from cell culture supernatant, the transplantation point and the chondrogenic potency of the cells can be ascertained by a person skilled in the art. For example, considering the cell culture condrogenic potency of MSC or chondrocytes which have lost their chondrogenic potency by expansion, these cells can be treated with growth factors to regain chondrogenic potency.

By virtue of this invention it is no longer necessary to excise cells before transplantation, for example by the FACS measuring method as described in U.S. Pat. App. Pub. No. 2003/0235813, filed Apr. 24, 2003.

All features disclosed in this specification, including any accompanying claims, abstract, and drawings, may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means for” performing a specified function, or “step for” performing a specific function, is not to be interpreted as a “means” or “step” clause as specified in 35 U.S.C. § 112, paragraph 6. In particular, the use of “step of” in the claims herein is not intended to invoke the provisions of 35 U.S.C. § 112, paragraph 6.

Although preferred embodiments of the present invention have been shown and described, various modifications and substitutions may be made thereto without departing from the spirit and scope of the invention. Accordingly, it is to be understood that the present invention has been described by way of illustration and not limitation.

What is claimed is:

1. A method for measuring the concentration of a marker chosen from the list of SERPINA1 and MMP3 from cell culture supernatant as secreted marker molecules from cells embedded in three-dimensional scaffolds.

2. The method of claim 1, wherein the concentrations of the marker molecules are correlated with the specific potency of the cells.

3. The method of claim 1 or claim 2, wherein the concentrations of SERPINA 1 or MMP3, or the combination of the concentrations are measured in the culture supernatant of chondrocytes or chondrocyte-like cells to determine chondrogenic potency.

4. The method of claim 3, wherein the condrocytes or chondrocyte-like cells are embedded in three dimensional scaffolds.

5. The method of claim 3, wherein the chondrocytes or chondrocyte-like cells are expanded under mono-layer conditions.

6. The method according to any one of claims 1 to 5, wherein chondrogenic potency is monitored in re-differentiation culture systems of chondrocytes loosing their chondrogenic potency.

7. The method according to any one of claims 1 to 5, wherein chondrogenic potency of chondrocyte-like cells in cell culture systems is monitored during differentiation in growth factor treated cultures.

8. The method according to any one of claims 1 to 6 for establishing a chondrongenic re-differentiation protocol for chondrocytes getting back chondrongenic potency.

9. The method of claim 7, for establishing a differentiation protocol for chondrocyte-like cells receiving chondrogenic potency.

10. The method according to any one of the preceding claims, wherein the chondrogenic potency of the chondrocytes or chondrocyte-like cells used in transplantation procedures for repairing cartilage defects is measured at the time of transplantation.
